(19) United States
(12) Patent Application Publication
(10) Pub. No.: US 2023/0277422 A1
(43) Pub. Date: Sep. 7, 2023

(54) COSMETIC COMPOSITION COMPRISING CAPSULES

(71) Applicants: GIVAUDAN SA, VERNIER (CH); IAMFLUIDICS HOLDING B.V., Enschede (NL)

(72) Inventors: Benedikt Christoph FLAMMER, Schaffhausen (CH); Celine DINANT, Saint-Hilaire-le-Petit (FR); Tom KAMPERMAN, Lichtenvoorde (NL); Sara TAHAN LATIBARI, Enschede (ML); Jenny Evelin PARRAGA MENESES, Losser (NL)

(21) Appl. No.: 18/005,874

(22) PCT Filed: Aug. 3, 2021
(86) PCT No.: PCT/EP2021/071703

§ 371 Date: Jan. 18, 2023

(30) Foreign Application Priority Data
Aug. 3, 2020 (NL) 2026204

(51) Int. Cl. A61K 8/11; A61Q 5/02

(52) U.S. Cl. CPC A61K 8/11; A61Q 5/02; A61K 2800/43; A61K 2800/524; A61K 2800/56; A61K 2800/48; A61K 2800/41

(57) ABSTRACT

A cosmetic composition comprising a plurality of capsules and a cosmetic base is provided.

The invention relates to a cosmetic composition comprising a plurality of capsules and a cosmetic base.

In many applications and markets, active compounds are (micro-) encapsulated to enhance shelf life, prevent degradation, increase solubility, or mask the taste of the active ingredient. In pharmaceutics, for instance, (micro-) encapsulation strategies can be used to increase the solubility and to control the delivery of active pharmaceutical ingredients in the body. For cosmetics, (micro-) encapsulation strategies can be used to protect active cosmetic ingredients against oxygen-, temperature-, moisture-, or light-induced degradation. In food, (micro-) capsules are used to increase the shelf life, or to mask the flavor of bad-tasting nutrients. Also, (micro-) encapsulation strategies can be used for the controlled release and/or prolonged prevalence of aromas and ethereal oils by counteracting fast evaporation of fragrances.

Soft and hard shell capsules are widely used within the pharmaceutical, health and food industry and have gained an acceptance as they present pharmaceutical, cosmetic and health products in a form that is readily administered, consumed, applied and/or digested by a user. These capsules are generally made up of a shell and an active core material. The shell may be formed of readily digested materials; for example, a soft gelatin capsule may comprise a mixture of gelatin, glycerol and water. Hard shell capsules generally comprise gelatin and water. Generally, soft and hard shell capsules are suitable for encapsulating a wide range of pharmaceutical and health products in the form of an emulsion.

Ascorbic acid (also known as vitamin C) is one of the most used natural reducing agents or antioxidants. This vitamin is well-known for its general essential protective role in health. It is a naturally occurring, organic compound, which also plays a significant biological role in anti-oxidation, anti-aging, anti-cancer, and immune regulation. Therefore, ascorbic acid is added to many products, for instance inter alia in food, pharmaceutical, cosmetics, and dermatologic substances. In dermatology, vitamin C is known for its association with collagen synthesis, as well as for its anti-oxidative nature, which ultimately reinforces the skin appearance by avoiding the appearance of fine lines or wrinkles in the skin, as well as by a skin whitening effect, by restoring skin elasticity, and by eliminating harmful oxygen species generated by ultraviolet light.

Ascorbic acid is easily degraded by oxidation, especially in aqueous solutions. Oxidation of ascorbic acid results in the formation of de-hydro-ascorbic acid, which is hypothesized to degrade into 2,3-diketogulonic acid. These reaction products have significantly less anti-oxidative properties and are characterized by a yellow to brown color. Consequently, many vitamin C-laden products in, for example, topical cosmetic creams, are characterized by a yellow to yellow/brown coloration, which indeed indicates that the vitamin C is, at least partially, oxidized and/or degraded and thus provides sub-optimal anti-oxidative properties.

Ascorbic acid degradation via oxidation or via other mechanisms is induced and affected by several physical and chemical factors, such as elevated temperature, (UV) light, moisture, pH value, and the presence of (dissolved) oxygen species. Due to this large variety of degradation factors, formulating stable formulations of ascorbic acid is not trivial. Several known stabilization or preservation methods have focused on the dehydration or chemical modification of ascorbic acid. Dried forms of ascorbic acid, however, are limited to application in equally dry formulations, such as tablets and powders. Many chemically modified forms of ascorbic acid that are intended to avoid degradation in moist environments have unnatural chemical structures that are biologically less active and therefore provide reduced benefits.

Numerous alternative attempts have been made to conquer these limitations and find more stable compositions comprising ascorbic acid, for example by dissolving an appropriate derivative in an oil environment. To that end, water soluble vitamins, such as the B group vitamins and ascorbic acid, are generally presented in the form of a emulsion in edible oil and encapsulated in a soft gelatin or hard shell capsule. Oils, such as soy bean oil, are generally used. This hydrophobic environment can effectively minimize the oxidation of particularly vitamin C by reducing the amounts of water and oxygen to which ascorbic acid is exposed.

The vitamins may be used on their own as the active ingredient, or in combination with herbal materials, such as bioflavanoids, rutin or with other vitamins. Ascorbic acid, for example, may be combined with other vitamins, such as B group vitamins, beta-carotene, vitamin D and vitamin E, or with minerals, such as trace elements of iron, calcium, magnesium or zinc. Soft gelatin capsules containing vitamins, such as ascorbic acid, may be used for a number of therapeutic and complementary medicine purposes, for example as a component in an anti-oxidant therapy in conjunction with beta-carotene and vitamin E.

Ascorbic acid and other water-soluble vitamins, such as the B group vitamins, however, have a certain solubility in the gelatin shell and can migrate from the core material to the shell if not completely insolubilized. Over time, the water soluble vitamin may enter or even cross the shell and may oxidize or react. In order to counteract this mechanism, international patent application WO 99/24021 discloses a method of immobilizing an encapsulated vitamin composition, according to which water soluble vitamin particles are coated with a material that is substantially insoluble in both the oily core material as well as in the shell of the capsule. The coated water soluble vitamin particles are of a size that is suitable for encapsulation in the form of an oily emulsion that provides the core of the capsule.

Albeit being soluble in oil or fat, the solubility of these ascorbic acid derivatives and other active compounds in a lipid environment generally is relatively poor as compared to their solubility in water. Moreover, the fat-soluble derivatives of ascorbic acid appear to have practical limitations, for example due to their rapid decomposition, poor solubility in aqueous formulations, and poor biological function and bio-activity. Coating particles of the active compound as thought by WO 99/24021 not only immobilizes the active compound, but moreover also deactivates the active compound. The coating needs to be removed to reactivate the active compound, for instance by chemical reaction in the gastrointestinal tract of the user.

It is therefore, inter alia, an object of the invention to provide an improved method of preservation of a reactive active compound, particularly a water soluble active compound, as well as a capsule and formulation containing such a compound, while maintaining the active compound in an active condition.

This object is solved by the cosmetic composition of the present invention as described below.

The present disclosure relates to a method of preservation of a reactive active compound, wherein said active compound is brought in a liquid and said liquid, containing said active compound, is encapsulated by a surrounding shell layer. The disclosure moreover relates to such a capsule, comprising a core encapsulated in a shell layer, wherein said core comprises a liquid containing an active compound, as well as to a formulation comprising a fluid material like a cream, lotion, gel, serum, cleanser, soap, shampoo, oil or clay that comprises a cosmetic, pharmaceutical, nutritious or organoleptic active compound.

In order to attain said aim, a method of the type described in the above paragraph is characterized in that said active compound is brought in a first liquid, in that said first liquid, containing said active compound, is brought in a second liquid that is substantially immiscible with said first liquid, that an emulsion is formed containing said first liquid and said second liquid, and in that said emulsion is encapsulated by a shell layer, in particular a solid shell layer, wherein both said first liquid and said shell layer are hydrophilic and said second liquid is hydrophobic.

A capsule of the type described in the above paragraph is characterized in that said liquid comprises an emulsion of a first liquid and a second liquid, said first and second liquid being substantially immiscible with one another and said first liquid comprising said active compound, wherein said core that comprises said emulsion is surrounded by a shell layer, in particular a solid shell layer, wherein said first liquid is hydrophilic, wherein said second liquid is hydrophobic, and wherein said shell layer is hydrophilic.

According to the invention, a formulation of the type as described in the above paragraph is characterized in that said fluid material comprises a plurality of capsules containing a suspension or emulsion comprising a hydrophilic first liquid and a hydrophobic second liquid that are immiscible with one another, containing said active compound in said first liquid, confined within a hydrophilic shell layer, in particular a solid shell layer.

The present invention provides, in particular, a cosmetic composition comprising a plurality of capsules and a cosmetic base.

According to the invention, the capsules comprise a core encapsulated in a shell layer, said core comprising a liquid containing a cosmetic active compound; and said liquid comprises an emulsion of a first liquid and a second liquid, said first and second liquid being substantially immiscible with one another.

Typically, a ratio of about 1:3 to about 1:1 of the first liquid to the second liquid is used, but higher or lower ratios would also be possible.

According to the invention, the cosmetic active compound is present in said first liquid and the core of said emulsion is surrounded by said shell layer.

Typically, the core makes up about 1-90% (v/v) of the capsule, more preferably about 10-50% (v/v).

The cosmetic active compound is selected from the group consisting of a vitamin and/or a salt and/or an ester thereof, a natural colorant, an energetic molecule, a cofactor, an antioxidant, an anti-acne agent, a whitening agent, an enzyme, a self-tanning reagent, a plant stem cell, a peptide, a polypeptide, a protein, a polysaccharide, a cooling agent, a warming or tingling agent, a liposoluble compound, a powder, a pigment, resveratrol, azelaic acid, ellagic acid and/or a salt and/or an ester thereof, fumaric acid and/or a salt and/or an ester thereof, rutin, ferulic acid, escin, sericoside, asiaticoside, madecassoside, salicylic acid, and mixtures thereof. It has been found that these cosmetic active compounds are particularly well stabilized by the capsules of the present invention.

The cosmetic base is selected from the group consisting of a cream, a lotion, a gel, an oil, a shampoo base, a shower gel, a hair conditioner, and a serum.

Accordingly, the active compound may readily be dissolved or otherwise brought (e.g. suspended or dispersed) in a first liquid and then emulsified with a second liquid that will then serve as a barrier between the small, dispersed, particularly colloid, droplets of said first liquid, containing the active compound, and the surrounding shell. This protective environment has proven to protect said active compound against degradation and counteracts migration of said active compound to and across the shell layer. Leakage of the active compound through the shell layer to an unprotected or less protected (e.g. oxidizing) environment that would otherwise lead to premature degradation may thereby be avoided or at least counteracted.

The method, capsule and cosmetic composition described herein are particularly useful for protecting active compounds with relatively low stability that might otherwise be prone to e.g. oxidation, degradation or other chemical modification. Thanks to the encapsulation, these active compounds may be protected against interaction with other components in a formulation, in particular a cosmetic composition, but also with the environment, e.g. oxygen in the air or light.

Another advantage of the method, capsule and cosmetic composition described herein is that active compounds, which are otherwise not very well soluble in a cosmetic base, may be encapsulated and, thus, become suitable for use in this particular cosmetic base. For instance, thanks to the encapsulation, oils may be used in aqueous formulations and water soluble actives may be used in oily formulations.

The capsules and compositions containing said capsules are also able to provide a sensorial benefit upon release of the active contained in the capsules, for instance upon breakage of the capsules. Examples of sensorial benefits include, but are not limited to, a change in texture (e.g. upon release of an oily active from an aqueous composition), smell (e.g. upon release of a fragrant active or a mixture of actives comprising a fragrance), color (e.g. upon release of a pigment or colorant), or other sensorial aspects (e.g. cooling, warming, heating, tingling or soothing).

Last but not least, the encapsulation as described herein may also be used to make a cosmetic composition visually more attractive, e.g. by including capsules that are visible to the eye or by ensuring that the composition is completely homogenous.

In an embodiment, the first liquid is a hydrophilic liquid and the second liquid is a hydrophobic liquid. Preferably, the shell layer is also hydrophilic.

In another embodiment, the first liquid is a hydrophobic liquid and the second liquid is a hydrophilic liquid. Preferably, the shell layer is also hydrophobic.

A particular embodiment of the method and capsule is characterized in that said first liquid comprises water. An aqueous liquid is non-toxic and in many cases very practical to process particularly when used in combination with a water soluble active compound.

Alternatively or in addition, said first liquid comprises a water-free solvent, particularly glycerine, glycerol or poly(ethylene glycol).

Similarly, a particular embodiment of the method and capsule is characterized in that said second liquid comprises at least one of organic (e.g. plant or animal) oils, waxes and fats, and particularly in that said second liquid is an oil selected from a group consisting of essential oils, ethereal oils, macerated oils, triglyceride and mixtures or derivatives thereof, and particularly comprises sunflower oil. Like water, also many of these naturally occurring oils and waxes are non-toxic and user-friendly. In this embodiment, the hydrophobic environment created by the second liquid forms an effective barrier between the hydrophilic first liquid and the hydrophilic shell layer to prevent the first liquid, containing the active compound, from leaking through the shell layer.

Particularly if applied in cosmetics and nutrients, ethereal, macerated and/or essential oils or waxes furthermore add favorable, pleasant organoleptic properties to the product. Examples of suitable organic lipophilic compounds are for instance:
plant oils, such as sunflower oil, corn oil, castor oil, palm oil, coconut oil, avocado oil, sweet almond oil, calophyllum oil, lanolin, sesame oil, olive oil, jojoba oil, soybean oil, cottonseed oil, rapeseed oil, peanut oil, flaxseed oil, borage oil and plant oil derivatives,
essential oils, such as immortelle, lavender, German chamomile, neroli, peppermint oil, rosemary, rose oil, tea tree oil, dwarf pine, juniper berry, roast chestnut extract, birch leaf extract, hayseed extract, ethyl acetate, camphor, menthol, rosemary extract, eucalyptus oil,
macerated oils,
fatty acids, such as stearic acid, palmitic acid, behenic acid, myristic acid, lauric acid and capric acid,
fatty acid derivatives, such as fatty acid esters with short chain alcohols, such as isopropyl myristate, isopropyl palmitate and isopropyl stearate and dibutyl adipate;
medium and long chain fatty acids and their esters with polyols, such as propylene glycol;
animal oils, such as tallow fat and marine oils, such as fish oils and seaweed oils or mixtures thereof;
nut oils, seed oils, waxes, such as paraffin wax, montan wax, glycol montanate, carnauba wax, paraffin wax, candelilla wax, beeswax, microcrystalline wax, ozocerite wax; and
triglyceride.

A particularly favorable stability is achieved in a preferred embodiment, wherein said second liquid comprises a fat or wax in a form of micro-particles, preferably of micro-particles having a size smaller than 1 mm. These fat or wax micro-particles are believed to further immobilize hydrophilic droplets of the first liquid, containing the active compound, within the second liquid.

In general, fats and waxes are solid or creamy (malleable) at room temperature. Advantage may be taken of this natural nature of waxes and compounds to further immobilize the active compound within the capsule. Accordingly, a special embodiment of the method and capsule according to the invention is characterized in that said second liquid is a fat or wax that is processed in a liquid condition to solidify or have solidified between room temperature and at least approximately human body temperature. These waxes or fats may be processed in a liquid form to be able to create capsules, for instance by using the method of a co-pending European patent application EP 3 436 188 A1, the subject matter of which is herewith incorporated by reference. Subsequently, the product may be stored at room temperature or below room temperature while the fat or wax contained in the capsule is in a solid state, thereby counteracting migration of both the active compound out of the capsule as well as of environmental compounds into the capsule. Applied in cosmetics or for pharmaceutical in or on the human body at body temperature, the wax will start to liquefy, having for instance a melting point just above 40° C., thereby mobilizing and subsequently releasing its active ingredient.

Therefore, in an embodiment, said second liquid comprises a fat or wax that is processed in a liquid condition to solidify below 40° C., particularly between room temperature and human body temperature.

Preferably, said liquid condition is attained without adversely affecting the condition of the (aqueous) first liquid containing the active compound. Therefore, in an embodiment, the fat or wax has a melting point below about 90° C. This avoids boiling and consequently disintegration of the aqueous first liquid into droplets that are also dispersed throughout the second liquid and moreover suppresses thermally induced degradation of the active compound. Suitable candidates for these wax or fat micro-particles include, for instance, montan wax, carnauba wax, glycol montanate, paraffin wax, candelilla wax, beeswax, microcrystalline wax and ozocerite wax, and mixtures thereof.

Therefore, in an embodiment, said fat or wax is selected from the group consisting of montan wax, carnauba wax, glycol montanate, paraffin wax, candelilla wax, beeswax, microcrystalline wax, ozocerite wax, and mixtures thereof.

In an embodiment, said second liquid comprises an organic oil selected from the group consisting of essential oils, ethereal oils, macerated oils, triglyceride and mixtures or derivatives thereof, and particularly comprises sunflower oil.

Preferably, the capsule is loaded with a sufficient amount of the active compound. To that end, a preferred embodiment of the method and capsule is characterized in that said active compound is dissolved in said first liquid. According to this embodiment, the active compound and first liquid are adapted to one another, such that the active compound will be readily dissolvable in said liquid. Particularly high amounts of active compound may be loaded in the first liquid without substantially influencing the viscosity of the fluid concerned and its process behavior.

In a further particular embodiment, the method and capsule are characterized in that said first liquid contains said active compound in a supersaturated condition. Super saturation is a solution that contains more of the dissolved active compound than could be dissolved by the solvent under normal circumstances. This allows for a further increase in the load of active compound in a capsule. Raising the temperature, pressure or volume of the liquid may allow more compound to be dissolved than possible at normal, lower values. By changing back these parameters to their normal, lower values at a faster rate than required for the compound to precipitate or crystalize, the solution may become in a supersaturated condition, containing an increased amount of the active compound.

The first liquid may be hydrophilic or hydrophobic, in which case the other, second liquid will be hydrophobic or hydrophilic respectively.

A specific embodiment of the method and capsule is characterized in that said first liquid is a hydrophilic solution, emulsion or suspension of said active compound, particularly an aqueous solution, while said second liquid is hydrophobic, wherein more particularly said shell layer is hydrophilic.

The hydrophilic first liquid may, however, in itself already be suspended in a hydrophobic third liquid that in turn is suspended in a hydrophilic fourth liquid, and so on, to form a multiple-phase emulsion or suspension in the second liquid that may contain multiple active compounds or ingredients in either a hydrophilic or hydrophobic phase of such multiple emulsion or suspension.

In all these cases, the hydrophobic second liquid forms a continuous phase that surrounds dispersed kernels of the hydrophilic first liquid ultimately containing the active compound. This hydrophobic continuous phase will from a barrier between the first liquid and the surrounding shell layer, particularly a shell layer that is likewise hydrophilic, to thereby shield and confine the dispersed phase formed by the first liquid within the capsule and the active compound within its surrounding kernel.

In an embodiment, the method and capsule are characterized in that said shell layer comprises a polymer network, in particular a solid polymer network, and more particularly an interpenetrating network of two or more cross-linked polymers.

In a specific embodiment, said polymer network comprises a hydrophilic polymer network, particularly comprising one or more poly-electrolytes or polysaccharides selected from the group consisting of agar, alginate, chitosan, dextran, poly(ethylene glycol), collagen, gelatin, hyaluronic acid, carrageenan, fibroin, fibronectin, poly-L-lysine (PLL), cellulose, graphene, polyethylenimine (PEI), poly(amidoamine) (PAA), dextran sulfate, silk, silk fibroin, pectin, Kappa-carrageenan, Iota-carrageenan, gellan gum, guar gum, tragacanth gum, xanthan gum, acacia gum, karaya gum, sodium carboxymethyl cellulose (S-CMC), and mixtures thereof; all of these as naturally derived materials and/or synthetically derived materials including recombinant proteins and/or derivatives of these materials. Preferably, the shell layer comprises alginate and/or agar and/or dextrose and/or gelatin, and in particular, alginate.

Particularly successful results were obtained in this respect with a further specific embodiment of the method and capsule, wherein said polymer network comprises a cross-linked or interpenetrating alginate network, particularly a cross-linked calcium-alginate network. The network may be further strengthened by incorporation of micro-particles and/or poly-electrolytes.

Therefore, a particularly preferred embodiment of the method and capsule is characterized in that bivalent cations are added to said first liquid, particularly by means of an electrolyte supplying magnesium ions and/or calcium ions, more particularly a calcium or magnesium salt solution providing a ionic calcium concentration of at least 0.001 M in said first liquid, preferably at least 0.01 M, more particularly between 0.1 M and 1.0 M, even more particularly between 0.1 M and 0.5 M. This supply of divalent cations, particularly divalent magnesium and/or calcium ions, happens to stabilize and strengthen the calcium-alginate network by preventing exhaustion of the cross linking calcium therein.

Optionally, the shell layer may be further stabilized, for instance by adding agar and/or a mineral (such as laponite) to the calcium-alginate network.

Alternatively, a further specific embodiment of the method and capsule is characterized in that said first liquid is a hydrophobic solution or suspension of said active compound, while said second liquid is hydrophilic, wherein more particularly said shell layer is hydrophobic. In this case, the continuous phase of the emulsion is formed by the hydrophilic second liquid, surrounding dispersed kernels of the hydrophobic first liquid containing the active compound. This hydrophilic continuous phase will form a barrier between the hydrophobic first liquid and the surrounding shell layer, particularly a shell layer that is likewise hydrophobic, to thereby shield and confine the first liquid to the capsule and the active compound to its kernel. In this case, a preferred embodiment of the method and capsule according to the invention is characterized in that said shell layer comprises a hydrophobic polymer network, particularly an interpenetrating network of two or more cross-linked polymers.

In general, the present invention is based on the recognition that a reactive or otherwise vulnerable active compound that is prone to degradation may effectively be preserved by bringing said compound in droplets of a first liquid and surrounding these droplets by a shielding second liquid, and that such a system may be provided by a suitable suspension or emulsion of both liquids, said second liquid forming the continuous phase of said emulsion. This system can be confined to a capsule of desired size by encapsulating an appropriate volume of said emulsion by a suitable shell layer.

As both liquids are in principle immiscible mutually, it is important to avoid phase separation and coalescence of the first liquid in the capsule. To that end, suitable surfactants, stabilizers and/or emulsifiers may be added to the system to counteract such separation. Such a surfactant may be:
a nonionic surfactant, such as polyoxyethylene sorbitan monolaureate (TWEEN®; e.g. TWEEN®-20), polyoxyethylene 23 lauryl ether (e.g. BRIJ®-35) or polyoxyethylated octyl phenol (e.g. TRITON®);
a zwitterionic surfactant, such as 3-((3-cholamidopropyl) dimethylammonio)-1-propane sulfonate (e.g. CHAPS®);
a cationic surfactant; or
an anionic surfactant, such as cholate, deoxycholate, sodium dodecyl sulfate, or TWEEN®-80.

As far as the method and capsule are concerned, a particularly stable emulsion is created in a further preferred embodiment of the method and capsule that is characterized in that said emulsion is stabilized by solid particles which adsorb onto an interface between said two liquids, and more particularly in that said solid particles are micro-particles or nano-particles that act as Pickering stabilizer.

These solid particles may be inorganic or organic.

A particular embodiment of the method and capsule is characterized in that said solid particles comprise micro-particles and/or nano-particles that are selected from a group of particles containing silver, gold, fullerene, silicon, aluminum, calcium carbonate, zinc, mica, titanium, copper, platinum, silicic acid, lithium, magnesium, iron, magnetic nanoparticles, nanotubes, protein, cellulose, clay or nano clay (in particular laponite clay), and mixtures thereof, including any of the oxidized forms of these materials, such as, e.g. silicon dioxide or silica.

Examples of inorganic nano- or micro-particles that may act as a Pickering stabilizer are:
Silicas, particularly fumed silica,
Metal oxides, like Fe₃O₄ nanoparticles, TiO₂, CuO,
Clays, particularly montmorillonite (MMT) and Laponites RD,
Layered double hydroxide (LDH), and
Carbons, like carbon nanotube (CNT), graphene oxide (GO), carbon black (CB).

Examples of organic nano or micro particles that may act as a Pickering stabilizer are:
Proteins, like bovine serum albumin coupled with PNIPAM,
Polysaccharide nanocrystals, like cellulose nanocrystals,
Chitin nanocrystals,
Starch nanocrystals,
Polymer particles, like poly(divinylbenzene-methacrylic acid) (P(DVB-MAA)) particles, polystyrene (PS) and poly(methyl methacrylate) (PMMA) nanoparticles,
Hydroxyapatite, and
Chitosan.

The Pickering stabilization may be further enhanced by an appropriate pre-treatment. In that respect, a further preferred embodiment of the method and capsule is characterized in that said solid particles are hydrophobized, particularly by functionalizing or coating with a chlorosilane or silanol, particularly (alkyl)chlorosilane, trimethylsilanol, dimethyldichlorosilane or poly(dimethylsiloxane).

In a particular embodiment, the solid particles comprise fumed silica nano-particles, preferably post-treated with dimethyldichlorosilane (Si(CH₃)₂Cl₂).

To further enhance the Pickering stabilization of the emulsion, electrostatically charged particles or polymers may be added to the first liquid having a polarity like that of the Pickering particles being used. Specifically, a polyanionic polymer or negatively charged glycosaminoglycans may be added to stabilize a Pickering emulsion based on negatively charged Pickering particles, like fumed silica nano-particles. Conversely, positively charged electrostatically charged particles, like chitosan, may be added to the first liquid to stabilize a Pickering emulsion that uses a positively charged Pickering agent. Particularly, said solid particles maybe charged negatively and said agent then may comprise a polyanionic polymer or a negatively charged glycosaminoglycan, more particularly hyaluronic acid, carageenan or acacia gum.

In view of the use of the capsules for human or animal use, a further preferred embodiment of the method and capsule is characterized in that said solid particles comprise micro-particles and/or nano-particles that are biocompatible and, in particular, biodegradable. As an example, said solid particles may comprise micro-particles and/or nano-particles that are food grade stabilizers, such as particles containing alginate, starch, gelatin, fatty acids and/or derivatives thereof.

These particles may be further enhanced if said solid particles comprise a biodegradable compound or are hydrophobized by functionalizing or coating with a biodegradable compound, particularly with a compound selected from the group consisting of amino acids, polyhydroxystearic acid, stearoyl glutamic acid, natural olive esters, jojoba ester, magnesium myristate, hydrogenated lecithin, lecithin, silica, isopropyl titanium triisostearate, dimethicone, methicone, and mixtures thereof.

For stabilizing the active compound and, hence, improving shelf-life, it may further be favorable to tweak and tune its chemical environment, in particular that of the first liquid. To that end, a further particular embodiment of the method and capsule is characterized in that an acid, a base and/or a buffer agent is added to said first and/or second liquid, maintaining a pH of said first and/or second liquid at a predetermined level that further aids in stabilizing said active compound.

In a particular embodiment, an acid, a base and/or a buffer agent selected from the group consisting of ascorbic acid, hyaluronic acid, a zwitterionic sulfonic acid buffering agent and mixtures thereof, more particularly 4-(2-hydroxyethyl)-1-piperazineethanesulfonic acid (HEPES), metaphosphoric acid, citric acid, perchloric acid, acetic acid and/or orthophosphoric acid is added.

Said pH value may, specifically, be held below a pH value of about 6-7 to promote an acidic condition of the first and/or second liquid.

Preferably, the acid, base and/or buffer agent is added to the hydrophilic liquid.

A particularly stable suspension or emulsion is obtained if ascorbic acid is added to said first liquid in a form of a micronized ascorbic acid powder, having a maximum particle size below 150 micron, particularly at a concentration of more than 25 wt %. Therefore, in an embodiment, ascorbic acid is added to said first liquid in a form of a micronized ascorbic acid powder, having a maximum particle size below 150 micron, particularly at a concentration of more than 25 wt %.

In principle, the capsules according to the invention can be realized in a wide range of dimensions. The application of the active compound has, however, been found particularly favorable if provided in the form of micro- or nano-capsules, having sub-micron to millimeter size.

Accordingly, a further preferred embodiment of the method according the invention is characterized in that said shell layer encloses a volume of between the order of one femtoliter and the order of one milliliter. For example, the shell layer may enclose a volume of less than one milliliter, thereby forming a micro-capsule.

Similarly, a further preferred embodiment of the capsule is characterized in that said core has a Feret diameter averaged over all directions of the order of between 0.5 μm and 10 mm, particularly between 10 μm and 10 mm, more particularly between 50 μm and 5 mm.

In this respect, capsules that are intended to be visible to the eye are preferably created having a diameter of between 500 μm and 5000 μm, whereas smaller micro-capsules may be created having a diameter between 50 μm and 500 μm.

Particularly, a shell layer thickness constitutes less than 25% of the diameter of said capsule and the core comprises said first liquid in a ratio of at least 1:100, particularly at least 1:10, more particularly at least 1:3, compared to said second liquid. Particularly, the first liquid forms a dispersed phase in a continuous phase that is formed by said second liquid and as such will not exceed the content of said second liquid. Said active compound constitutes preferably between 10% and 50% by weight of said first liquid.

In principle, a wide variety of active ingredients may be processed with the method according to the invention and supplied in the form of one or more capsules according to the invention. However, the present invention is particularly useful for active compounds that would be unstable or prone to rapid degradation when exposed to (excessive) moisture or air.

The invention proves especially beneficial in certain specific embodiments, in which said active compound is selected from the group consisting of pharmaceutical agents, fragrances, cosmetic agents, flavors, nutrients and mixtures thereof, wherein more particularly said active compound is selected from the group consisting of vitamins, anti-oxidants, proteins and/or derivatives thereof, and mixtures thereof.

As an example, the method and capsule may be used in embodiments, wherein said active compound comprises at least one vitamin, particularly a vitamin that is selected from the group consisting of thiamine, riboflavin, nicotinic acid, pantothenic acid, pyridoxine, biotin, folic acid, cyanocobalamin, lipoic acid, ascorbic acid, lecithin, glycyrrhizin acid, retinol, retinol palmitate, tocopherol, tocopherol acetate, salicylic acid, benzoyl peroxide, and azelaic acid and/or derivatives thereof, and mixtures thereof, more particularly ascorbic acid and/or derivatives thereof.

The capsules may in particular be used for cosmetic purposes, for instance for skin therapy or skin protection. To that end, a special embodiment of the capsule according to the invention is characterized in that said shell layer is configured to break or rupture under a mechanical load, particularly a manually applied mechanical load, more particularly by squeezing or chewing. The configuration of the shell layer in this case promotes the release of its active contents when being applied, particularly rubbed out, onto the skin.

The shell layer may be provided with a coloring, particularly colored particles, more particularly water-insoluble pigments, even more particularly UV-absorbing and/or UV-reflecting pigments.

In an embodiment, the capsules are characterized by a substantially spherical shape.

The size of the capsule may be adapted to the intended use or application. For instance, the size may be chosen to be relatively large, to obtain capsules that are visible to the eye.

In an embodiment, the capsules have a Feret diameter averaged over all directions of between 1 μm and 10 mm, more particularly between 10 μm and 10 mm, and even more particularly between 50 μm and 5 mm.

The thickness of the shell layer may be adapted to the intended use or application. In particular, the thickness of the shell layer should be chosen such that a sufficient stability of the capsule is achieved. At the same time, it is important that the active compound contained in the capsules is released at the desired time and under the desired circumstances.

In an embodiment, the shell layer constitutes less than 1% by volume of said capsule.

The amounts of the first and second liquid, as well as of the active compound, may also be adapted to the intended use or application.

In an embodiment, the first liquid constitutes about 20-60% by volume of said capsule, more preferably about 25-50%, for example about 40%.

In an embodiment, the active compound constitutes about 1-80% by weight of said capsule, more preferably about 1-50%, for example about 10-30%.

In a further aspect, the present invention also provides a formulation comprising a plurality of the capsules of the present invention, in particular a cosmetic formulation.

Throughout this disclosure, the terms “composition” and “formulation” are used as synonyms and each term may replace the other.

The formulation of the present invention comprises a fluid material like a cream, lotion, gel, serum, cleanser, soap, shampoo, oil or clay that comprises a cosmetic, pharmaceutical, nutritious or organoleptic active compound, characterized in that said fluid material comprises a plurality of capsules containing a suspension comprising a hydrophilic first liquid and a hydrophobic second liquid that are immiscible with one another, containing said active compound in said first liquid, confined within a hydrophilic solid shell layer.

The formulation of the present invention is particularly suitable for use in cosmetics.

Therefore, the present invention provides a cosmetic composition comprising a plurality of capsules and a cosmetic base, wherein the capsules comprise a core encapsulated in a shell layer, said core comprising a liquid containing a cosmetic active compound and said shell layer comprising calcium alginate, wherein said liquid comprises an emulsion of a first liquid and a second liquid, said first and second liquid being substantially immiscible with one another, wherein said cosmetic active compound is present in said first liquid and the core of said emulsion is surrounded by said shell layer, wherein the cosmetic active compound is selected from the group consisting of a vitamin and/or a salt and/or an ester thereof, a natural colorant, an energetic molecule, a cofactor, an antioxidant, an anti-acne agent, a whitening agent, an enzyme, a self-tanning reagent, a plant stem cell, a peptide, a polypeptide, a protein, a polysaccharide, a cooling agent, a warming or tingling agent, a liposoluble compound, a powder, a pigment, resveratrol, azelaic acid, ellagic acid and/or a salt and/or an ester thereof, fumaric acid and/or a salt and/or an ester thereof, rutin, ferulic acid, escin, sericoside, asiaticoside, madecassoside, salicylic acid, and mixtures thereof, and wherein the cosmetic base is selected from the group consisting of a cream, a lotion, a gel, an oil, a shampoo base, a shower gel, a hair conditioner, and a serum.

Serums are cosmetic formulations containing a high level of active compounds, which may have different physical forms, e.g. a cream, lotion or gel with increased active content.

By choosing a suitable base, it is possible to make the capsules “float” in the composition, i.e. to avoid deposition (at the bottom) or creaming (at the top). To this end, the exact composition of the base and/or the capsules may be adjusted, e.g. to obtain a similar density and/or to prevent deposition/creaming by mechanical means, e.g. by increasing the viscosity.

In an embodiment, the cosmetic active compound is selected from the group consisting of:
a vitamin or vitamin derivative selected from the group consisting of ascorbic acid, an ascorbic acid salt or ester (e.g. ascorbyl palmitate or ascorbyl acetate), retinol, a retinyl ester (e.g. retinyl palmitate), thiamine, riboflavin, nicotinic acid, a nicotinic acid salt or ester, pantothenic acid, a pantothenic acid salt or ester, pyridoxine, biotin, folic acid, a folic acid salt or ester, cyanocobalamin, lipoic acid, a lipoic acid salt or ester, and niacinamide; in particular ascorbic acid and/or a salt thereof;
a natural colorant selected from the group consisting of an anthocyanin (e.g. cyanidin, peonidin, malvidin, delphinidin, petunidin, pelargonidin), curcumin, chlorophyll, lycopene, beta-carotene, capsanthin, capsorubin, and spirulina;
an energetic molecule or cofactor selected from the group consisting of adenosine triphosphate (ATP), adenosine diphosphate (ADP), nicotinamide adenine dinucleotide (NAD+ or NADH), nicotinamide adenine dinucleotide phosphate (NADP+ or NADPH), coenzyme Q10, superoxide dismutase (SOD), and glutathione;
an antioxidant selected from the group consisting of a polyphenol, thiol-based component, a sulphite and derivatives thereof, tocopherol, carnosic acid, a tocotrienol, and a flavonoid;
an anti-acne and/or whitening agent selected from the group consisting of benzoyl peroxide, glycyrrhizin acid, and a glycyrrhizin acid derivative;
an enzyme selected from the group consisting of a lipase, a protease, an esterase, papain, and bromelain;
a spider silk protein;
a polysaccharide selected from the group consisting of hyaluronic acid and/or a salt and/or a derivative thereof (e.g. sodium hyaluronate, hyaluronic acid acetate or a cationized hyaluronic acid), xanthan gum, and rhizobium gum;
a cooling agent selected from the group consisting of menthol and a menthol derivative (e.g. a menthyl carboxamide);
a warming or tingling agent selected from the group consisting of alpha hydroxy sanshool, capsaicin, spilanthol, ginger oil, and black pepper oil;
a liposoluble compound selected from the group consisting of jojoba oil, cranberry oil, rosephip oil, argan oil, kendi oil, grapeseed oil, bilberry seed oil, coffee oil, apricot kernel oil, buriti oil, chia seed oil, camelina oil, tsubaki oil, tea tree oil, karanja oil, moringa oil, tea seed oil, ungurahui oil, nymaplung oil, marula oil, bisabolol, and canabidiol;
a powder selected from the group consisting of zinc oxide, titanium dioxide, charcoal, and bamboo powder;
and mixtures thereof.

Depending on the intended cosmetic application, the cosmetic formulation will have a different composition. Thus, the composition of the cosmetic formulation, or at least of its major components, should be adapted to the intended use.

In the cosmetic bases described herein, water is typically used q.s.p. (i.e. the amount remaining to have 100%), unless otherwise indicated.

In an embodiment, the cosmetic base is a cream comprising an emulsifier, e.g. Ceteth-20 and/or Cetyl Alcohol and/or Glyceryl Stearate and/or PEG-75 Stearate and/or Steareth-20; fatty acids, e.g. medium-chain fatty acids (such as medium-chain triglycerides), stearic acid, palmitic acid, Cetearyl Wheat Straw Glycosides and/or Cetearyl Alcohol (e.g. Xyliance®), sucrose ester (such as sucrose palmitate, sucrose distearate, and/or sucrose tristearate); a preservative, e.g. phenoxyethanol and/or paraben and/or a paraben blend; and water; and optionally further comprising one or more of a humectant, e.g. glycerin; a fragrance; a pigment; and a color.

In an embodiment, the cream comprises about 3-10% of the emulsifier and less than 1% of the preservative.

Emulium® Delta (ex. Gattefossé), which is a mixture of Ceteth-20 and Cetyl Alcohol and Glyceryl Stearate and PEG-75 Stearate and Steareth-20, is an example of a suitable emulsifier.

Xyliance®), which contains a mixture of Cetearyl Wheat Straw Glycosides and Cetearyl Alcohol, is an example of a suitable emulsifier.

Mygliol® 812 N (ex. Caesar & Loretz GmbH), which contains medium-chain triglycerides, is an example of suitable fatty acids.

Phenonip™ XB (ex. Clariant), which is a liquid paraben blend, is an example of a suitable preservative.

In an embodiment, the cosmetic base is a gel comprising a thickener, e.g. xanthan gum; a preservative, e.g. phenoxyethanol and/or paraben and/or a paraben blend; and water; and optionally further comprising one or more of a humectant, e.g. glycerin, a fragrance, a pigment; and a color.

In an embodiment, the gel comprises about 0.5-1.5% of the thickener and less than 1% of the preservative.

Phenoxetol™ (ex. Clariant), which is based on phenoxyethanol, is an example of a suitable preservative.

In an embodiment, the cosmetic base is an oil, in particular a jellified oil, comprising an emollient, e.g. coco-glycerides and/or octyldodecanol and/or C12-15 alkyl benzoate; a thickener, e.g. silica and/or cellulose and/or a cellulose derivative, such as ethyl cellulose, dextrin palmitate, dextrin myristate, dextrin palmitate ethylhexanoate, bentonite; and optionally further comprising one or more of xanthan gum; acrylate copolymers (e.g. carbomers); a fragrance; a pigment; and a color.

In an embodiment, the oil comprises about 10-80% of the emollient and about 1-10% of the thickener.

Myritol® 331 (ex. BASF), which is a modified coconut oil, is an example of a suitable emollient with medium spreadability and very good emulsifiability. Eutanol® G (ex. BASF), which contains octyldodecanol, is another suitable emollient.

Ethocel™ Standard 100 Premium (ex. Dow), an ethyl cellulose polymer, is an example of a suitable thickener. It exhibits dimensional stability, heat stability and compatibility with plasticizers, waxes, oils and other resins. It may be used at a concentration of about 1-8% and is able to provide a transparent gel with most oils.

Octyldodecanol, C12-C15 alkylbenzoate and/or cocoglycerides may be used to solubilize Ethocel™ or other cellulose based thickeners. DUB B1215 (ex. Stearinerie Dubois) is an example of a suitable C12-C15 alkylbenzoate.

Silica is typically used at a concentration of up to about 7-8% in order to suspend the capsules in the gel while preserving transparency. An example of a suitable silica is Aerosil® R972 (ex. Evonik), a fumed silica aftertreated with dimethyldichlorosilane (DDS).

Typically, the (jellified) oil base contains little to no water, i.e. it is essentially or completely anhydrous. At the same time, the capsules comprised in the composition may contain water.

In an embodiment, the cosmetic base is a shampoo base comprising a surfactant, e.g. sodium laureth sulfate and/or cocamidopropyl betaine; a preservative, e.g. phenoxyethanol; and water; and optionally further comprising one or more of a conditioner, e.g. hydroxypropyl guar and/or hydroxypropyl guar hydroxypropyltrimonium chloride; a pH modulator, e.g. an acid, such as citric acid, or a base; a viscosity controller and/or bulking agent, e.g. sodium chloride or another salt; a fragrance; a pigment; and a color.

In an embodiment, the shampoo comprises about 10-40% of the surfactant, less than 1% of the preservative, and about 0.1-0.5% of the conditioner.

SLES Texapon NSO (sodium laureth sulfate) and Dehyton Kcos (cocamidopropyl betaine) are examples of suitable surfactants. They may be provided as a solution in water, optionally containing other ingredients, e.g. NaCl.

Hydroxypropyl guar and/or hydroxypropyl guar hydroxypropyltrimonium chloride may be used to provide hair conditioning, improve foam density, improve wet and dry combability, and/or provide a pleasant soft dry hair feel. Jaguar C162 is an example of such a material.

In an embodiment, the cosmetic base is a shampoo base comprising a surfactant, e.g. coco-glucoside and/or decyl-glucoside and/or a quillaja saponaria wood extract; an emollient, e.g. glyceryl oleate; a preservative, e.g. sodium benzoate and/or potassium sorbate; and water; and optionally further comprising one or more of a conditioner, e.g. xanthan gum; a pH modulator, e.g. an acid, such as lactic acid, or a base; a fragrance; a pigment; and a color.

In an embodiment, the shampoo comprises about 10-30% of the surfactant and less than 1% of the preservative.

Lamesoft® PO 65 (ex. BASF), which contains a mixture of coco glucoside and glyceryl oleate, is an example of a suitable ingredient, which provides natural moisturizing and acts as a natural lipid layer enhancer.

Sapnov™ vegan (ex. Naturex), a quillaja saponaria wood extract, is a suitable non-ionic surfactant with a foam booster property.

Plantacare® 818 UP (ex. BASF), which contains coco-glucoside, is another example of a suitable surfactant.

Oramix™ NS 10 (ex. SEPPIC), which contains decyl-glucoside, is another example of a suitable surfactant.

XGF FEDCS-PC (ex. AMI/Jungbunzlauer), is an example of a suitable xanthan gum.

Purac HS 90 is an example of a suitable lactic acid.

Some particularly well suited cosmetic bases are listed in the table below (all concentrations are indicated in % weight per weight):

| Base | Composition |
|---|---|
| Cream | 5% of a mixture of Ceteth-20, Cetyl Alcohol, Glyceryl Stearate, PEG-75 Stearate |
| | and Steareth-20 (e.g. Emulium Delta); 12% medium-chain triglycerides (e.g. |
| | Mygliol 812 N); 0.4% phenoxyethanol and/or paraben and/or a paraben blend |
| | (e.g. Phenonip XB); 1% glycerin; and 80.6% water |
| Gel | 1% xanthan gum; 3% glycerin; 0.5% phenoxyethanol (e.g. Phenoxetol); and |
| | 80.6% water |
| Jellified oil | 25.5% C12-15 alkyl benzoate (e.g. DUB B1215); 20% coco-glycerides (e.g. |
| | Myritol 331); 43.5% octyldodecanol (e.g. Eutanol G); 3% ethyl cellulose (e.g. |
| | Ethocel Std 100 Premium; and 7% silica (e.g. Aerosil 200) |
| Shampoo | 28.93% sodium laureth sulfate (e.g. SLES Texapon NSO 27% in water); 12% |
| | cocamidopropyl betaine (e.g. Dehyton Kcos 30% in water); 0.6% |
| | phenoxyethanol; 0.2% hydroxypropyl guar and/or hydroxypropyl guar |
| | hydroxypropyltrimonium chloride (e.g. Jaguar C162); 0.07% citric acid; 0.5% |
| | sodium chloride; 0.2% fragrance; and 56.5% water |
| Sulfate-free | 1% xanthan gum (e.g. XGF FEDCS-PC); 0.2% coco-glucoside (e.g. Plantacare |
| shampoo | 818 UP); 20% decyl-glucoside (e.g. Oramix NS 10); 7% coco-glucoside/glyceryl |
| | oleate (e.g. Lamesoft PO 65); 0.2% quillaja saponaria wood extract (e.g. Sapnov |
| | vegan); 0.3% sodium benzoate; 0.2% potassium sorbate; 2% lactic acid (e.g. |
| | Purac HS 90); and 62.1% water |

The amount of capsules contained in the cosmetic composition of the present invention may be adapted to the intended use or application. In particular, more or less concentrated formulations are possible.

The cosmetic composition may comprise about 0.1 to about 95% of capsules, preferably about 0.1 to about 50% of capsules, more preferably about 0.1 to about 10% of capsule, and most preferably about 0.5 to about 5% of capsules, e.g. about 1% of capsules.

The size of the capsules may also be adapted to the intended use or application, in particular depending on whether or not it is desired that the capsules are visible to the eye.

In an embodiment, the capsules have an average diameter of about 70 to about 5000 μm, more preferably of about 1000 to about 3000 μm, most preferably about 1500 to about 2500 μm. A typical size for capsules that are visible to the eye is about 2000 μm.

In an embodiment, the capsules are visible to the eye.

In an embodiment, the formulation comprises capsules, the shell layer of which is configured to break or rupture under a mechanical load, particularly a manually applied mechanical load, more particularly by squeezing or chewing.

For certain applications, it may be desirable that the capsules have a certain color. To this end, the shell layer may comprise a colorant and/or pigment. In particular, the shell layer may comprise a colorant and/or pigment selected from the group consisting of an anthocyanin (e.g. cyanidin, peonidin, malvidin, delphinidin, petunidin, pelargonidin), curcumin, chlorophyll, lycopene, beta-carotene, capsanthin, capsorubin, spirulina, zinc oxide, titanium dioxide, iron oxide, charcoal, and mixtures thereof. Advantageously, for easier incorporation into the shell layer, said colorant and/or pigment is provided in the form of particles with a size of less than 50 μm in order to avoid clogging or blocking of nozzles.

The cosmetic composition of the present invention may, in addition to the capsules described herein above, comprise one or more further cosmetic actives. Said further cosmetic active(s) may be the same or different from the cosmetic active compound contained in the capsules. In particular, the cosmetic composition of the present invention may comprise one or more further hair care or skin care active.

Examples of further actives that may be included in the cosmetic composition of the present invention include, but are not limited to, conditioning agents, such as hydrolysed collagen, vitamin E, panthenol, panthenyl ethyl ether, hydrolyzed keratin, proteins, plant extracts, and nutrients; hair-fixative polymers, such as amphoteric, non-ionic, cationic, and anionic fixative polymers, and silicone grafted copolymers; preservatives, such as benzyl alcohol, methyl paraben, propyl paraben, and imidazolidinyl urea; pH adjusting agents, such as glutamic acid, citric acid, sodium citrate, succinic acid, phosphoric acid, lactic acid, sodium hydroxide, and sodium carbonate; salts in general, such as potassium acetate and sodium chloride; coloring agents; hair oxidizing (bleaching) agents, such as hydrogen peroxide, perborate, and persulfate salts; hair reducing agents, such as thioglycolates; fragrances; and sequestering agents, such as disodium ethylenediamine tetra-acetate; ultraviolet and infrared screening and absorbing agents, such as octyl salicylate; moisturizers, such as hyaluronic acid or hyaluronic acid derivatives; hydrating agents; anti-ageing actives; soothers; cleansing agents; whitening agents; concealers; coolants; deodorants; antiperspirants; disinfectants; and mixtures thereof.

Although the invention has been described herein with reference to merely a limited number of explanatory embodiments, it should be understood that the invention is by no means limited to those examples. On the contrary many more variations and embodiments are feasible to a skilled person within the framework of the present invention without requiring him or her to exercise any inventive skill. Particularly, further components other than the first fluid may also contain one or more active compounds, notably also the (hydrophobic) second fluid. The first fluid may be emulsified directly with the second fluid or may find itself in an emulsion with one or more further fluids to be jointly emulsified or mixed with the second fluid. Also, the second fluid may itself consist of an emulsion with a further fluid. Each fluid may be used as a carrier for one or more specific active compounds or ingredients.

The invention is described in further detail with reference to a number of exemplary embodiments and accompanying drawings:

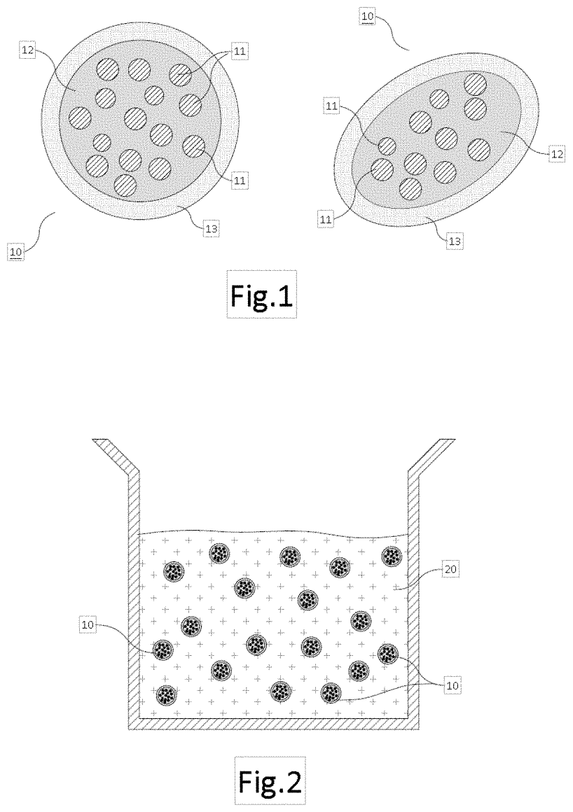

FIG. 1 shows a specific example of a capsule according to the invention; and

FIG. 2 shows an embodiment of a formulation according to the invention.

It is noted that the figures are drawn purely schematically and not necessarily to a same scale. In particular, certain dimensions may have been exaggerated to a more or lesser extent to aid the clarity of any features. Similar parts are generally indicated by a same reference numeral throughout the figures.

The capsules 10 that are shown in FIG. 1 are manufactured using a method according to the invention and resemble the protective nature that is provided by the invention to a vulnerable active compound that would otherwise be prone to oxidation or other degradation when exposed to ambient air, for instance. The active compound comprises for example ascorbic acid, being readily soluble in water. By dissolving micronized powder, for instance having a modal particle size of below around 75-100 micron, a large quantity of for instance between 20 and 25 wt % of ascorbic acid may be brought in an aqueous hydrophilic phase 11. The hydrophilic phase 11 is brought in a suspension with a hydrophobic continuous phase 12, for instance containing a plant oil like sunflower oil. Solid nano-clay particles of laponite clay may be added to the mixture to act as a Pickering stabilizer to stabilize the emulsion, counteracting coalescence of the aqueous phase droplets 11 and phase separation. The emulsion comprised of small dispersed hydrophilic droplets 11 surrounded by a hydrophobic continuous phase 12 is encapsulated within a hydrophilic solid shell layer 13, for instance created by a solidified cross-linked hydrophilic calcium alginate network. The capsules 10 typically enclose a volume of less than one milliliter, thereby forming a micro-capsule containing said suspension comprising said first liquid 11 and said second liquid 12, protecting said active compound in said first liquid.

The formulation of FIG. 2 comprises a plurality of micro-capsules 10 as shown in FIG. 1, which contain an appropriate active compound. The capsules are distributed in a suitable fluid material 20 like a cream, lotion, gel, serum, cleanser, soap, shampoo, oil or clay to provide a cosmetic composition that expresses the desired properties of the active compound.

The present invention is further illustrated by means of the following non-limiting examples:

### EXAMPLE 1: PRESERVATION OF ASCORBIC ACID (AA) VIA ENCAPSULATION IN OIL-FILLED CALCIUM-ALGINATE CAPSULES

Ascorbic acid was encapsulated in oil-filled alginate capsules. Specifically:

Creation of a Preserved AA Laden Sample.

50% (w/v) AA sodium salt (sodium L-ascorbate) was dissolved in water, which was then emulsified in sunflower oil. A stable surfactant-free water-in-oil (w/o) emulsion was generated by shaking and ultrasonically treating degassed water and oil solutions.

The AA-laden w/o emulsion was kept at 70° C. while it was jetted with a flow rate of 13 ml/min from the center nozzle of a coaxial nozzle assembly (OD=1.6 mm and ID=0.41 mm) as disclosed in the aforementioned co-pending European patent application EP 3 436 188 A1, the subject matter of which in respect to the capsule generation is herewith incorporated by reference.

Concurrently, a 0.5% (w/v) sodium alginate in water solution (WAKO, 1% ˜80-120 cP) of room temperature was jetted from the outer nozzle of the same coaxial nozzle assembly at a flow rate of 55 ml/min, resulting in a compound jet consisting of AA-laden w/o emulsion encapsulated by a sodium alginate solution. The coaxial nozzle was modulated, using a vibrating element at a frequency of approximately 150 Hz, which caused the controlled breakup of the compound jet into a stream of substantially mono-disperse, i.e. substantially uniformly sized, compound droplets with typically a coefficient of variation in size or diameter of less than 10%.

Via a so called ‘in-air microfluidics’ method, as described in the aforementioned co-pending application (EP 3 436 188), the droplet stream was combined with an intact, i.e. uninterrupted jet, of a 0.2 M calcium in water solution, resulting in the formation of compound hydrogel capsules consisting of a calcium-alginate shell layer filled with a core of AA-laden w/o emulsion. These hydrogel capsules were stored in water and incubated for 6 weeks at 40° C.

(ii) Creation of a Non-Preserved AA Laden Reference Sample.

For comparison purposes, a non-preserved AA control sample was created by dissolving 0.5% (w/v) AA in water, resulting in the same final concentration as that of the preserved sample. Also this non-preserved AA in water solution was incubated for 6 weeks at 40° C.

(iii) Comparison

Comparing a color change of the preserved sample to that of the similarly incubated (6 weeks at 40° C.) reference solution showed a brown coloring of the reference solution that is typical for the oxidation product of AA, while the preserved sample showed no significant coloring. This revealed that the encapsulation of AA in oil-filled capsules, according to the invention, reduces coloring, indicating suppression or even prevention of AA degradation.

### EXAMPLE 2: BOTH PHYSICAL ENCAPSULATION AND THE PRESENCE OF CALCIUM

To investigate the mechanism of AA preservation via encapsulation in calcium-alginate shells, three conditions were compared:
(a) AA preserved as described in example 1, under (i);
(b) Non-preserved control: same as (a), but using a 0.2 M sodium chloride solution instead of a 0.2 M calcium chloride solution, which will prevent the formation of gelled calcium-alginate capsules and thus the encapsulation of the AA-laden w/o emulsion;
(c) Non-preserved control: same as (a), but the 0.2 M calcium chloride jet was not impacted with the compound droplets prior to collection in the collector bath, which prevents the in-flight formation of stable calcium-alginate capsules and, thus, the encapsulation of the AA-laden w/o emulsion.

The concentration of AA in all conditions was at least 5% (w/v) in the w/o emulsions and at least 0.5% (w/v) in the final sample volumes.

Comparison of the color change after incubation at 40° C. for 4 weeks showed no significant coloring of the preserved sample (a), comprising AA-laden hydrogel capsules having a calcium cross-linked alginate shell layer, while sample (b) showed significant browning after 4 weeks and sample (c) showed slight coloring. This revealed that the presence of divalent calcium ions has a preserving effect on AA.

### EXAMPLE 3: OIL-FILLED CAPSULES USING PICKERING EMULSIFIERS

Fumed silica nanoparticles post-treated with dimethyl-dichlorosilane (DDS) (Evonik, Aerosil R972) were added to the core of the capsules by dispersing 2% (w/v) of nano-particles in the oil phase. The oil containing hydrophobized silica particles was then processed following the same method as described in example 1, except that a supersaturated AA-solution containing 100% (w/v) was used. In this example, calcium carbonate (CaCO₃) was added to the dispersed AA-laden phase before emulsification to load the capsules with excess CaCO₃ that will aid in maintaining a stable calcium-alginate shell over time.

The thus created AA-laden silica-oil filled calcium-alginate capsules had a final AA concentration in the w/o emulsion (i.e. the core compound) of at least 10% (w/v), a final CaCO₃ concentration in the w/o emulsion of 2% (w/v) and a final hydrophobized silica concentration in the w/o emulsion of 2% (w/v). The capsules were washed two times with demineralized water and incubated in demineralized water at 40° C. for 23 days.

After 23 days, the samples showed no significant coloring, revealing an effective preservation of both the AA active compound in the dispersed phase as well as of the w/o emulsion itself and the surrounding calcium-alginate shell layer.

### EXAMPLE 4: OIL-FILLED CAPSULES USING PICKERING EMULSIFIERS IN CORE AND ADDITIVES IN SHELL

Fumed silica nano-particles were post-treated with dimethyl-dichlorosilane (DDS) (Evonik, Aerosil R972) and added to the core of the capsules by dispersing 4% (w/v) of nano-particles in an oil phase. The oil containing these hydrophobized silica particles was processed following the same method as described in example 1 together with an aqueous solution of ascorbic acid to form an emulsion, except that a saturated L-ascorbic acid fine powder containing 40% (w/v) was used for the solution.

Furthermore, 0.5% laponite XL21XR nano-clay was added to the 0.5% alginate phase in the shell during the encapsulation. This created AA-laden silica-oil filled calcium-alginate/laponite capsules, having a final AA concentration in the w/o emulsion (i.e. the core compound) of at least 10% (w/v), and a final hydrophobized silica concentration in the w/o emulsion of 4% (w/v).

The capsules were washed two times with demineralized water and incubated in demineralized water, 0.2 M CaCl₂+10 wt % ethanol solution, clear hand gel, shampoo, and body lotion, respectively, at 40° C. for 4 weeks. After 4 weeks, the samples showed no significant coloring compared to a similar sample containing the same emulsion without encapsulation and L-ascorbic acid bulk solution in water. This revealed an effective preservation of both the AA active compound in the dispersed phase as well as of the w/o emulsion itself by the surrounding calcium-alginate shell layer.

### EXAMPLE 5: AGAR REINFORCED ENCAPSULATED CAPSULES

Similar capsules were prepared using the same method as described in example 4, but additionally using 0.5 wt % agar solution for forming the shell composition. This resulted in ascorbic acid containing emulsion capsules within a 1% laponite XL21XR/0.5% alginate/0.5% agar solid shell layer. These capsules were stored for 4 weeks in water, shampoo, body lotion and 0.2 M CaCl₂+10% EtOH, respectively, and were found to show hardly any coloring compared to the bare emulsion (i.e. unencapsulated ascorbic acid emulsion) in the same base.

### EXAMPLE 6: LAYER-BY-LAYER COATING OF THE SOLID SHELL

Capsules were prepared in a similar way to that of example 4, but with coating by a layer using electrostatically interaction of layer-by-layer method (LBL). The alginate shell was coated by a positively charged biopolymer, such as chitosan, to enhance the barrier property and stability of L-ascorbic acid. Chitosan was applied as a polycationic polymer to ionically crosslink the alginate shell.

Initially, a stable 5 wt % chitosan stock solution was prepared in aid of 1% (v/v) hydrochloric acid (HCl) while stirring for several hours at 50° C. Afterwards, the 5 wt % chitosan solution was neutralized by adjusting the pH to about 6-7 by adding sodium hydroxide (NaOH) solution. Then, the neutralized solution was diluted to obtain a 0.5 wt % chitosan solution to be used in the coating process.

Vitamin C encapsulated capsules in an alginate shell were coated with a chitosan/alginate bilayer. First, the capsules were rinsed with water and then incubated in a 0.5 wt % neutralized chitosan solution while stirring slowly for 15 min. The incubated beads were filtered and rinsed with water, then incubated and stirred slowly in 0.5 wt % of alginate solution for another 15 min. To avoid the aggregation of capsules during static incubation, slow movement (stirring) was applied while incubating the beads. To achieve a denser coating, a salt solution (NaCl solution) was applied to coat the same capsules by ionic crosslinking of chitosan/alginate. The purpose of using salts was to ensure attaining a stable thickness by alternating deposition. A 0.8 M NaCl solution was applied in all incubation and rinsing steps.

### EXAMPLE 7: IMPROVED STABILIZATION USING SOLIDIFIED SOLID WAX PARTICLES

Similar capsules were prepared according to the method as described in example 4, but instead of fumed silica nano-particles, 2 wt % micronized carnauba wax (Microcare 350, Micro Powder Inc.) was added to the oil phase (i.e., the second liquid).

Alternatively, a sample was prepared containing an oil phase with 4 wt % hydrophobized fumed silica, as well as 2 wt % micronized carnauba wax.

The capsules were then incubated for 0.5, 1, 2, 5, 10, and 20 min at 40, 60, 75, or 90° C. 5 min incubation at 90° C. appeared optimal for melting the micronized wax particles in the capsules in order to render the suspension more stable after the wax had again solidified in the oily phase.

The capsules were tested for ascorbic acid stability by incubating them in water, shampoo, body lotion and 0.2M CaCl₂+10% EtOH, at 40° C. After this treatment, the capsules showed hardly any yellow coloring as compared to the same particles that were mechanically disrupted at the start of the stability test, indicating an improved chemical stability (i.e., preservation) of the encapsulated ascorbic acid within capsules containing a Pickering and wax stabilized emulsion of the dispersed ascorbic acid aqueous phase.

### EXAMPLE 8: ELECTROSTATICALLY LOADING THE FIRST LIQUID

An aqueous phase from the below list was added to 6 wt % Aerosil R972 to act as a Pickering stabilizer in sunflower oil. Three homogenization cycles using T25 Ultra-Turrax with S25N-25F dispersing element were applied to form a Pickering emulsion: 1 cycle of 2 min at 10,000 rpm and 2 cycles of 1 min at 10,000 rpm. In between the cycles, the emulsion was mixed manually to ensure a total incorporation of the phases.
1. Water
2. Water+1 wt % NaCl
3. Water+HCl (pH 2)
4. Water+20 wt % hyaluronic acid
5. Water+20 wt % hyaluronic acid+1 wt % NaCl
6. Water+2 wt % carrageenan
7. Water+2 wt % acacia gum
8. Water+2 wt % acacia gum+1 wt % NaCl
9. Water+1 wt % chitosan

The resulting emulsion of samples 4-7 appeared to be significantly more stable than the other samples. This supports the assumption that the water-in-oil Pickering emulsion based on negatively charged Aerosil R972 as the Pickering agent is further stabilized by the addition of polyanionic polymers or (negatively charged) glycosaminoglycans, notably by hyaluronic acid, carrageenan or acacia gum, to the aqueous phase.

### EXAMPLE 9: STABILITY TESTING OF CAPSULES IN COSMETIC FORMULATIONS

Ten different types of capsules in six different cosmetic bases were subjected to stability testing. Samples were kept at 4° C., 20° C., and 40° C.

### Capsules

The following ten types of capsules were tested:

| # | Capsules |
|---|---|
| 1 | 0.5% calcium-alginate matrix capsules, suspended in 0.2M CaCl₂ + 10% EtOH |
| 2 | 0.5% calcium-alginate matrix capsules with iron oxide yellow pigment, suspended in 0.2M |
| | CaCl₂ + 10% EtOH |
| 3 | 0.5% calcium-alginate + 0.5% agar matrix capsules with iron oxide yellow pigment, |
| | suspended in 0.2M CaCl₂ + 20% EtOH |
| 4 | 0.5% calcium-alginate matrix capsules with iron oxide yellow pigment + CaCO₃ |
| | microparticles, suspended in 0.2M CaCl₂ + 10% EtOH |
| 5 | 0.5% calcium-alginate matrix capsules with iron oxide yellow pigment + Laponite XL21 XR, |
| | suspended in 0.2M CaCl₂ +10% EtOH |
| 6 | 0.5% calcium-alginate + 0.5% agar matrix capsules with iron oxide yellow pigment + |
| | Laponite XL21 XR, suspended in 0.2M CaCl₂ + 20% EtOH |
| 7 | 0.5% calcium-alginate matrix capsules with chlorophyll in a sunflower oil core, suspended in |
| | 0.2M CaCl₂ + 10% EtOH |
| 8 | 0.5% calcium-alginate with 0.5% Laponite XL21 XR + 0.5% agar-based capsules with |
| | chlorophyll in a sunflower oil core, suspended in 0.2M CaCl₂ + 20% EtOH |
| 9 | 0.5% calcium-alginate with 0.5% agar-based capsules with chlorophyll in a sunflower oil |
| | core, suspended in 0.2M CaCl₂ + 20% EtOH |
| 10 | 0.5% calcium-alginate + Laponite XL21 XR based capsules with an emulsion-filled core |
| | containing 40% L-ascorbic acid in water + 4% Aerosil ® R972 in sunflower oil, suspended in |
| | 0.2M CaCl₂ + 20% EtOH |

### Cosmetic Compositions

The above ten capsule types 1-10 were tested in the following six cosmetic formulations, the compositions of which are described in the six tables below (concentrations indicated in % weight by weight):

A. Classical Shampoo

| | A1 | A2 | A3 | A4 | A5 | A6 | A7 | A8 | A9 | A10 |
|---|---|---|---|---|---|---|---|---|---|---|
| SLES Texapon | 28.93 | 28.93 | 28.93 | 28.93 | 28.93 | 28.93 | 28.93 | 28.93 | 28.93 | 28.93 |
| NSO 27% | | | | | | | | | | |
| Dehyton Kcos 30% | 12 | 12 | 12 | 12 | 12 | 12 | 12 | 12 | 12 | 12 |
| Phenoxyethanol | 0.6 | 0.6 | 0.6 | 0.6 | 0.6 | 0.6 | 0.6 | 0.6 | 0.6 | 0.6 |
| Fragrance | 0.2 | 0.2 | 0.2 | 0.2 | 0.2 | 0.2 | 0.2 | 0.2 | 0.2 | 0.2 |
| Water | 36.5 | 36.5 | 36.5 | 36.5 | 36.5 | 36.5 | 36.5 | 36.5 | 36.5 | 36.5 |
| Jaguar C162 | 0.2 | 0.2 | 0.2 | 0.2 | 0.2 | 0.2 | 0.2 | 0.2 | 0.2 | 0.2 |
| Water | 20 | 20 | 20 | 20 | 20 | 20 | 20 | 20 | 20 | 20 |
| Citric acid | 0.07 | 0.07 | 0.07 | 0.07 | 0.07 | 0.07 | 0.07 | 0.07 | 0.07 | 0.07 |
| NaCl powder qs | 0.5 | 0.5 | 0.5 | 0.5 | 0.5 | 0.5 | 0.5 | 0.5 | 0.5 | 0.5 |
| visco 5000 cps | | | | | | | | | | |
| Capsule Type #1 | 1 | | | | | | | | | |
| Capsule Type #2 | | 1 | | | | | | | | |
| Capsule Type #3 | | | 1 | | | | | | | |
| Capsule Type #4 | | | | 1 | | | | | | |
| Capsule Type #5 | | | | | 1 | | | | | |
| Capsule Type #6 | | | | | | 1 | | | | |
| Capsule Type #7 | | | | | | | 1 | | | |
| Capsule Type #8 | | | | | | | | 1 | | |
| Capsule Type #9 | | | | | | | | | 1 | |
| Capsule Type #10 | | | | | | | | | | 1 |

B. Sulfate-Free Shampoo

| | B1 | B2 | B3 | B4 | B5 | B6 | B7 | B8 | B9 | B10 |
|---|---|---|---|---|---|---|---|---|---|---|
| XGF FEDCS-PC | 1 | 1 | 1 | 1 | 1 | 1 | 1 | 1 | 1 | 1 |
| (=xanthan gum) | | | | | | | | | | |
| Water | 59.5 | 59.5 | 59.5 | 59.5 | 59.5 | 59.5 | 59.5 | 59.5 | 59.5 | 59.5 |
| Plantacare 818 UP | 20 | 20 | 20 | 20 | 20 | 20 | 20 | 20 | 20 | 20 |
| (=coco-glucoside) | | | | | | | | | | |
| Oramix NS 10 | 7 | 7 | 7 | 7 | 7 | 7 | 7 | 7 | 7 | 7 |
| (=decyl-glucoside) | | | | | | | | | | |
| Lamesoft PO 65 = | 6 | 6 | 6 | 6 | 6 | 6 | 6 | 6 | 6 | 6 |
| (coco-glucoside, | | | | | | | | | | |
| glyceryl oleate) | | | | | | | | | | |
| Sapnov vegan | 2 | 2 | 2 | 2 | 2 | 2 | 2 | 2 | 2 | 2 |
| Water | 1 | 1 | 1 | 1 | 1 | 1 | 1 | 1 | 1 | 1 |
| Sodium benzoate | 0.3 | 0.3 | 0.3 | 0.3 | 0.3 | 0.3 | 0.3 | 0.3 | 0.3 | 0.3 |
| Potassium sorbate | 0.2 | 0.2 | 0.2 | 0.2 | 0.2 | 0.2 | 0.2 | 0.2 | 0.2 | 0.2 |
| Purac HS 90 | 2 | 2 | 2 | 2 | 2 | 2 | 2 | 2 | 2 | 2 |
| (=lactic acid) | | | | | | | | | | |
| Capsule Type #1 | 1 | | | | | | | | | |
| Capsule Type #2 | | 1 | | | | | | | | |
| Capsule Type #3 | | | 1 | | | | | | | |
| Capsule Type #4 | | | | 1 | | | | | | |
| Capsule Type #5 | | | | | 1 | | | | | |
| Capsule Type #6 | | | | | | 1 | | | | |
| Capsule Type #7 | | | | | | | 1 | | | |
| Capsule Type #8 | | | | | | | | 1 | | |
| Capsule Type #9 | | | | | | | | | 1 | |
| Capsule Type #10 | | | | | | | | | | 1 |

C. Cream

| | C1 | C2 | C3 | C4 | C5 | C6 | C7 | C8 | C9 | C10 |
|---|---|---|---|---|---|---|---|---|---|---|
| Emulium Delta | 5 | 5 | 5 | 5 | 5 | 5 | 5 | 5 | 5 | 5 |
| Miglyol | 12 | 12 | 12 | 12 | 12 | 12 | 12 | 12 | 12 | 12 |
| Water | 80.6 | 80.6 | 80.6 | 80.6 | 80.6 | 80.6 | 80.6 | 80.6 | 80.6 | 80.6 |
| Phenonip XB | 0.4 | 0.4 | 0.4 | 0.4 | 0.4 | 0.4 | 0.4 | 0.4 | 0.4 | 0.4 |
| Glycerin | 1 | 1 | 1 | 1 | 1 | 1 | 1 | 1 | 1 | 1 |
| Capsule Type #1 | 1 | | | | | | | | | |
| Capsule Type #2 | | 1 | | | | | | | | |
| Capsule Type #3 | | | 1 | | | | | | | |
| Capsule Type #4 | | | | 1 | | | | | | |
| Capsule Type #5 | | | | | 1 | | | | | |
| Capsule Type #6 | | | | | | 1 | | | | |
| Capsule Type #7 | | | | | | | 1 | | | |
| Capsule Type #8 | | | | | | | | 1 | | |
| Capsule Type #9 | | | | | | | | | 1 | |
| Capsule Type #10 | | | | | | | | | | 1 |

D. Gel

| | D1 | D2 | D3 | D4 | D5 | D6 | D7 | D8 | D9 | D10 |
|---|---|---|---|---|---|---|---|---|---|---|
| Xanthan gum | 1 | 1 | 1 | 1 | 1 | 1 | 1 | 1 | 1 | 1 |
| Glycerin | 3 | 3 | 3 | 3 | 3 | 3 | 3 | 3 | 3 | 3 |
| Water | 94.5 | 94.5 | 94.5 | 94.5 | 94.5 | 94.5 | 94.5 | 94.5 | 94.5 | 94.5 |
| Phenoxethol | 0.5 | 0.5 | 0.5 | 0.5 | 0.5 | 0.5 | 0.5 | 0.5 | 0.5 | 0.5 |
| Capsule Type #1 | 1 | | | | | | | | | |
| Capsule Type #2 | | 1 | | | | | | | | |
| Capsule Type #3 | | | 1 | | | | | | | |
| Capsule Type #4 | | | | 1 | | | | | | |
| Capsule Type #5 | | | | | 1 | | | | | |
| Capsule Type #6 | | | | | | 1 | | | | |
| Capsule Type #7 | | | | | | | 1 | | | |
| Capsule Type #8 | | | | | | | | 1 | | |
| Capsule Type #9 | | | | | | | | | 1 | |
| Capsule Type #10 | | | | | | | | | | 1 |

E. Jellified Oil

| | E1 | E2 | E3 | E4 | E5 | E6 | E7 | E8 | E9 | E10 |
|---|---|---|---|---|---|---|---|---|---|---|
| DUB B1215 = C12-15 | 25.5 | 25.5 | 25.5 | 25.5 | 25.5 | 25.5 | 25.5 | 25.5 | 25.5 | 25.5 |
| Alkyl benzoate | | | | | | | | | | |
| Myritol 331 = Cocoglycerides | 20 | 20 | 20 | 20 | 20 | 20 | 20 | 20 | 20 | 20 |
| Eutanol G = Octyldodecanol | 43.5 | 43.5 | 43.5 | 43.5 | 43.5 | 43.5 | 43.5 | 43.5 | 43.5 | 43.5 |
| Ethocel Std 100 | 3 | 3 | 3 | 3 | 3 | 3 | 3 | 3 | 3 | 3 |
| Premium = Ethylcellulose | | | | | | | | | | |
| Aerosil 200 = Silica | 7 | 7 | 7 | 7 | 7 | 7 | 7 | 7 | 7 | 7 |
| Capsule Type #1 | 1 | | | | | | | | | |
| Capsule Type #2 | | 1 | | | | | | | | |
| Capsule Type #3 | | | 1 | | | | | | | |
| Capsule Type #4 | | | | 1 | | | | | | |
| Capsule Type #5 | | | | | 1 | | | | | |
| Capsule Type #6 | | | | | | 1 | | | | |
| Capsule Type #7 | | | | | | | 1 | | | |
| Capsule Type #8 | | | | | | | | 1 | | |
| Capsule Type #9 | | | | | | | | | 1 | |
| Capsule Type #10 | | | | | | | | | | 1 |

F. Hand Sanitizer

| | F1 | F2 | F3 | F4 | F5 | F6 | F | F8 | F9 | F10 |
|---|---|---|---|---|---|---|---|---|---|---|
| Carbopol Ultrez 20 | 0.2 | 0.2 | 0.2 | 0.2 | 0.2 | 0.2 | 0.2 | 0.2 | 0.2 | 0.2 |
| Water | 20.25 | 20.25 | 20.25 | 20.25 | 20.25 | 20.25 | 20.25 | 20.25 | 20.25 | 20.25 |
| Triethanolamine | | | | | | | | | | |
| sq pH 6.5 | | | | | | | | | | |
| EtOH 96% | 75 | 75 | 75 | 75 | 75 | 75 | 75 | 75 | 75 | 75 |
| Phenoxethol | 0.5 | 0.5 | 0.5 | 0.5 | 0.5 | 0.5 | 0.5 | 0.5 | 0.5 | 0.5 |
| Fresh Hydration | 0.05 | 0.05 | 0.05 | 0.05 | 0.05 | 0.05 | 0.05 | 0.05 | 0.05 | 0.05 |
| 68 EAE08472/00 | | | | | | | | | | |
| Vegetal glycerin | 3 | 3 | 3 | 3 | 3 | 3 | 3 | 3 | 3 | 3 |
| rapeseed | | | | | | | | | | |
| Capsule Type #1 | 1 | | | | | | | | | |
| Capsule Type #2 | | 1 | | | | | | | | |
| Capsule Type #3 | | | 1 | | | | | | | |
| Capsule Type #4 | | | | 1 | | | | | | |
| Capsule Type #5 | | | | | 1 | | | | | |
| Capsule Type #6 | | | | | | 1 | | | | |
| Capsule Type #7 | | | | | | | 1 | | | |
| Capsule Type #8 | | | | | | | | 1 | | |
| Capsule Type #9 | | | | | | | | | 1 | |
| Capsule Type #10 | | | | | | | | | | 1 |

### Procedures

The cosmetic compositions were prepared by first preparing the respective base and then adding the capsules at room temperature under very gentle stirring with a spatula.

The viscosity of the cosmetic compositions was determined using a Brookfield DVIII ultra spindle E or cylinder 3 and speed 12.

The pH was measured at 20° C.

### Results

The results of the stability testing are compiled in the following tables (compositions that were unstable at the beginning of the test (D0) were not tested further and are listed below the respective tables):

### A. Classical Shampoo

| | | A2 | A3 | A5 | A8 | A9 | A10 |
|---|---|---|---|---|---|---|---|
| D0 | Viscosity | 1380 | 1270 | 1150 | 1510 | 280 | 1350 |
| | [mPa · s] | | | | | | |
| | pH | 5.1 | 5.12 | 5.15 | 4.98 | 5.04 | 5.17 |
| | Aspect | Sedimentation, but | | Sedimentation, | Capsules at the surface | | Sedimentation, |
| | | capsules intact | | but capsules | | | but capsules |
| | | | | intact | | | intact |
| 28 d at | Aspect | OK | Smaller | OK | OK | OK | OK |
| 4° C. | | | capsules | | | | |
| | Smell | OK | OK | OK | OK | OK | PK |
| 28 d at | Aspect | OK | Smaller | OK | Capsules | Capsules | Beginning |
| 4° C. | | | capsules | | less green | less green | Lysis |
| | Smell | OK | OK | OK | OK | OK | OK |
| 28 d at | Aspect | OK | Smaller | OK | Capsule | Capsule | Pale yellow |
| 4° C. | | | capsules | | broken and | broken and | and lysis of |
| | | | | | sedimentation | green at | capsules |
| | | | | | | the surface | |
| | Smell | OK | OK | OK | OK | OK | oxidated |

Unstable at D0: A1, A4, A6, A7

### B. Sulfate-Free Shampoo

| | | B1 | B2 | B3 | B4 | B5 |
|---|---|---|---|---|---|---|
| D0 | Viscosity | 6540 | 6830 | 6490 | 6870 | 6840 |
| | [mPa · s] | | | | | |
| | pH | 5.42 | 5.38 | 5.35 | 5.35 | 5.36 |
| | Aspect | Homogenous capsules in suspension | | | | |
| 28 d at | Aspect | OK | OK | OK | OK | OK |
| 4° C. | Smell | OK | OK | OK | OK | OK |
| 28 d at | Aspect | 2-3 capsules | OK | OK | 2-3 capsules | OK |
| 4° C. | | broken | | | broken | |
| | Smell | OK | OK | OK | OK | OK |
| 28 d at | Aspect | Beginning of | OK | Beginning of | Broken | OK |
| 4° C. | | sedimentation | | sedimentation | capsules and | |
| | | and smaller | | and smaller | sedimentation | |
| | | capsules | | capsules | | |
| | Smell | OK | OK | OK | OK | OK |

| | | B6 | B7 | B8 | B9 | B10 |
|---|---|---|---|---|---|---|
| D0 | Viscosity | 7180 | 5730 | 6750 | 6150 | 6540 |
| | [mPa · s] | | | | | |
| | pH | 5.36 | 5.36 | 5.35 | 5.34 | 5.33 |
| | Aspect | Homogenous capsules in suspension | | | | |
| 28 d at | Aspect | OK | Capsule | Small halo | Small halo | OK |
| 4° C. | | | deformation | around | around | |
| | | | | capsules | capsules | |
| | Smell | OK | OK | OK | OK | OK |
| 28 d at | Aspect | OK | Capsule | Small halo | Small halo | OK |
| 4° C. | | | deformation | around | around | |
| | | | and less | capsules, | capsules, | |
| | | | green | about ½ | about ½ | |
| | | | | capsules | capsules | |
| | | | | broken | broken | |
| | Smell | OK | OK | OK | OK | OK |
| 28 d at | Aspect | Clear capsules | Smaller | Small halo | Small halo | Yellowing |
| 4° C. | | on top and | broken | around | around | of capsules |
| | | white capsules | capsules | capsules, | capsules, | and base |
| | | at the bottom | and release | about ½ | about ½ | |
| | | | of dye | capsules | capsules | |
| | | | | broken | broken | |
| | Smell | OK | OK | OK | OK | OK |

### C. Cream

| | | C1 | C2 | C3 | C4 | C5 |
|---|---|---|---|---|---|---|
| D0 | Viscosity | 12300 | 15300 | 13300 | 13400 | 13400 |
| | [mPa · s] | | | | | |
| | pH | 4.98 | 5.04 | 5.20 | 5.52 | 5.66 |
| | Aspect | Homogenous capsules in suspension | | | | |
| 28 d at | Aspect | OK | OK | OK | OK | OK |
| 4° C. | Smell | OK | OK | OK | OK | OK |
| 28 d at | Aspect | OK | OK | OK | OK | OK |
| 4° C. | Smell | OK | OK | OK | OK | OK |
| 28 d at | Aspect | OK | OK | OK | OK | OK |
| 4° C. | Smell | OK | OK | OK | OK | OK |

| | | C6 | C7 | C8 | C9 | C10 |
|---|---|---|---|---|---|---|
| D0 | Viscosity | 10000 | 16000 | 18300 | 10100 | 9200 |
| | [mPa · s] | | | | | |
| | pH | 4.80 | 5.00 | 4.68 | 5.11 | 4.87 |
| | Aspect | Homogenous capsules in suspension | | | | |
| 28 d at | Aspect | OK | OK | OK | OK | OK |
| 4° C. | Smell | OK | OK | OK | OK | OK |
| 28 d at | Aspect | OK | OK | OK | OK | OK |
| 4° C. | Smell | OK | OK | OK | OK | OK |
| 28 d at | Aspect | Smaller | OK | OK | OK | Capsules |
| 4° C. | | capsules | | | | yellowing |
| | Smell | OK | OK | OK | OK | OK |

### D. Gel

| | | D1 | D2 | D3 | D4 | D5 |
|---|---|---|---|---|---|---|
| D0 | Viscosity | 4250 | 3960 | 4120 | 4790 | 4510 |
| | [mPa · s] | | | | | |
| | pH | 5.89 | 5.97 | 6.25 | 7.99 | 6.22 |
| | Aspect | Homogenous capsules in suspension | | | | |
| 28 d at | Aspect | Capsule | Light capsule | Capsule | | Light capsule |
| 4° C. | | agglomerates | agglomerates | agglomerates | | agglomerates |
| | Smell | OK | OK | OK | OK | OK |
| 28 d at | Aspect | Capsule | Light capsule | Capsule | | Light capsule |
| 4° C. | | agglomerates | agglomerates | agglomerates | | agglomerates |
| | Smell | OK | OK | OK | OK | OK |
| 28 d at | Aspect | Capsule | Light capsule | Capsule | | Light capsule |
| 4° C. | | agglomerates | agglomerates | agglomerates | | agglomerates |
| | Smell | OK | OK | OK | OK | OK |

| | | D6 | D7 | D8 | D9 | D10 |
|---|---|---|---|---|---|---|
| D0 | Viscosity | 3040 | 3150 | 3810 | 3710 | 3850 |
| | [mPa · s] | | | | | |
| | pH | 6.29 | 6.27 | 6.03 | 6.22 | 6.37 |
| | Aspect | Homogenous capsules in suspension | | | | |
| 28 d at | Aspect | Capsules | Shell | ½ shell | | OK |
| 4° C. | | broken | disappearance | disappearance | | |
| | Smell | OK | OK | OK | OK | OK |
| 28 d at | Aspect | Smaller | Shell | ½ shell | | OK |
| 4° C. | | capsules | disappearance | disappearance | | |
| | Smell | OK | OK | OK | OK | OK |
| 28 d at | Aspect | OK | Shell | ½ shell | | Pale |
| 4° C. | | | disappearance | disappearance | | yellow |
| | | | | | | capsules |
| | Smell | OK | OK | OK | OK | OK |

### E. Jellified Oil

| | | E1 | E2 | E3 | E4 | E5 | E6 |
|---|---|---|---|---|---|---|---|
| D0 | Viscosity | 18000 | 18600 | 1880 | 18400 | 18040 | 20100 |
| | [mPa · s] | | | | | | |
| | Aspect | Limpid and homogenous capsules in suspension | | | | | |
| 28 d at | Aspect | OK | OK | OK | OK | OK | OK |
| 4° C. | Smell | OK | OK | OK | OK | OK | OK |
| 28 d at | Aspect | OK | OK | Smaller capsules | | 1 smaller | OK |
| 4° C. | | | | at the surface | | capsule and | |
| | | | | with a beginning | | beginning of | |
| | | | | of sedimentation | | sedimentation | |
| | Smell | OK | OK | OK | OK | OK | OK |
| 28 d at | Aspect | Smaller capsules at the surface with a beginning of | | | | | |
| 4° C. | | sedimentation, hazy aspect at the bottom | | | | | |
| | Smell | OK | OK | OK | OK | OK | OK |

| | | E7 | E8 | E9 | E10 |
|---|---|---|---|---|---|
| D0 | Viscosity | 18100 | 18500 | 19000 | 18500 |
| | [mPa · s] | | | | |
| | Aspect | Limpid and homogenous capsules in suspension | | | |
| 28 d at | Aspect | OK | OK | OK | OK |
| 4° C. | Smell | OK | OK | OK | OK |
| 28 d at | Aspect | OK | OK | OK | OK |
| 4° C. | Smell | OK | OK | OK | OK |
| 28 d at | Aspect | Less green | Less green | Pale green | Yellow smaller |
| 4° C. | | at the | at the | capsules with | capsules with |
| | | surface, | surface | a beginning of | a beginning of |
| | | hazy at | | sedimentation, | sedimentation, |
| | | the bottom | | hazy aspect at | hazy aspect at |
| | | | | the bottom | the bottom |
| | Smell | OK | OK | OK | OK |

### F. Hand Sanitizer

The tested capsules were incompatible with the tested base.

### Application to Skin

Compositions were applied to the skin (on the hand) after 1 month at 20° C. The following was observed:
Compositions A5 and A10 left no residue.
The capsules of composition A2 broke upon application, but there were some residues.
The capsules of composition A3 broke in the formula when trying to pick one capsule.
Compositions B1-B10 left no residue.
Composition B2 was slower to penetrate the skin than B1 and B3-B10.
The capsules in compositions C1-C4 and C6 did not break upon application to the skin.
The capsules in compositions C5, C9 and C10 did break upon application, but left a residue.
The capsules in compositions C7 and C8 did break upon application.
For compositions D1-D3, D6, D7, D9 and D10, there was no residue.
Compositions D4, D5, and D8 left a light residue.
The capsules in compositions E1-E6 did not break upon application to the skin.
The capsules in compositions E7-E10 did break upon application, but left a residue.

### CONCLUSIONS

The following observations were made:
All capsules were compatible with and stable in the tested cream base.
The hand sanitizer base tested was not compatible with the tested capsules.
No release of the yellow pigment was observed, except for the shampoo base A.
Chlorophyll was released in all bases, except for the cream base.
The yellow pigment was stable in the gel base.
Chlorophyll-containing capsules lost their shell in the gel base.
For yellow pigment in shampoo base A, only compositions A2, A3, and A4 were stable.
In all bases tested, capsules 2 (+agar) and 5 (Laponite XL21+agar) were the most stable capsules.
Yellowing was observed for more or less all capsules containing vitamin C in all bases.

Although the invention has been described herein before with reference to merely a limited number of explanatory embodiments, it should be understood that the invention is by no means limited to those examples. On the contrary, many more variations and embodiments are feasible to a skilled person within the framework of the present invention without requiring him or her to exercise any inventive skill. Particularly, further components other than the first liquid may also contain one or more active compounds, notably also the hydrophobic second liquid and/or the shell layer.

The first liquid may be emulsified directly with the second liquid or may find itself in an emulsion with one or more further liquid to be jointly emulsified or mixed with the second liquid. Also, the second liquid may itself consist of an emulsion with a further liquid. Each fluid may be used as a carrier for one or more specific active compounds or ingredients.

1. Cosmetic composition comprising a plurality of capsules and a cosmetic base,
wherein the capsules comprise a core encapsulated in a shell layer, said core comprising a liquid containing a cosmetic active compound,
wherein said liquid comprises an emulsion of a first liquid and a second liquid, said first and second liquid being substantially immiscible with one another,
wherein said cosmetic active compound is present in said first liquid and the core of said emulsion is surrounded by said shell layer,
wherein the cosmetic active compound is selected from the group consisting of a vitamin and/or a salt and/or an ester thereof, a natural colorant, an energetic molecule, a cofactor, an antioxidant, an anti-acne agent, a whitening agent, an enzyme, a self-tanning reagent, a plant stem cell, a peptide, a polypeptide, a protein, a polysaccharide, a cooling agent, a warming or tingling agent, a liposoluble compound, a powder, a pigment, resveratrol, azelaic acid, ellagic acid and/or a salt and/or an ester thereof, fumaric acid and/or a salt and/or an ester thereof, rutin, ferulic acid, escin, sericoside, asiaticoside, madecassoside, salicylic acid, and mixtures thereof, and
wherein the cosmetic base is selected from the group consisting of a cream, a lotion, a gel, an oil, a shampoo base, a shower gel, a hair conditioner, and a serum.

2. Cosmetic composition according to claim 1, wherein the cosmetic active compound is selected from the group consisting of:
a vitamin or vitamin derivative selected from the group consisting of ascorbic acid, an ascorbic acid salt or ester (e.g. ascorbyl palmitate or ascorbyl acetate), retinol, a retinyl ester (e.g. retinyl palmitate), thiamine, riboflavin, nicotinic acid, a nicotinic acid salt or ester, pantothenic acid, a pantothenic acid salt or ester, pyridoxine, biotin, folic acid, a folic acid salt or ester, cyanocobalamin, lipoic acid, a lipoic acid salt or ester, and niacinamide; in particular ascorbic acid and/or a salt thereof;
a natural colorant selected from the group consisting of an anthocyanin (e.g. cyanidin, peonidin, malvidin, delphinidin, petunidin, pelargonidin), curcumin, chlorophyll, lycopene, beta-carotene, capsanthin, capsorubin, and spirulina;
an energetic molecule or cofactor selected from the group consisting of adenosine triphosphate (ATP), adenosine diphosphate (ADP), nicotinamide adenine dinucleotide (NAD+ or NADH), nicotinamide adenine dinucleotide phosphate (NADP+ or NADPH), coenzyme Q10, superoxide dismutase (SOD), and glutathione;
an antioxidant selected from the group consisting of a polyphenol, thiol-based component, a sulphite and derivatives thereof, tocopherol, carnosic acid, a tocotrienol, and a flavonoid;
an anti-acne and/or whitening agent selected from the group consisting of benzoyl peroxide, glycyrrhizin acid, and a glycyrrhizin acid derivative;
an enzyme selected from the group consisting of a lipase, a protease, an esterase, papain, and bromelain;
a spider silk protein;
a polysaccharide selected from the group consisting of hyaluronic acid and/or a salt and/or a derivative thereof (e.g. sodium hyaluronate, hyaluronic acid acetate or a cationized hyaluronic acid), xanthan gum, and rhizobium gum;
a cooling agent selected from the group consisting of menthol and a menthol derivative (e.g. a menthyl carboxamide);
a warming or tingling agent selected from the group consisting of alpha hydroxy sanshool, capsaicin, spilanthol, ginger oil, and black pepper oil;
a liposoluble compound selected from the group consisting of jojoba oil, cranberry oil, rosephip oil, argan oil, kendi oil, grapeseed oil, bilberry seed oil, coffee oil, apricot kernel oil, buriti oil, chia seed oil, camelina oil, tsubaki oil, tea tree oil, karanja oil, moringa oil, tea seed oil, ungurahui oil, nymaplung oil, manila oil, bisabolol, and canabidiol;
a powder selected from the group consisting of zinc oxide, titanium dioxide, charcoal, and bamboo powder;
and mixtures thereof.

3. Cosmetic composition according to claim 1, wherein the cosmetic base is a cream comprising an emulsifier, e.g. Ceteth-20 and/or Cetyl Alcohol and/or Glyceryl Stearate and/or PEG-75 Stearate and/or Steareth-20; fatty acids, e.g. medium-chain fatty acids (such as medium-chain triglycerides), stearic acid, palmitic acid, Cetearyl Wheat Straw Glycosides and/or Cetearyl Alcohol, sucrose ester (such as sucrose palmitate, sucrose distearate, and/or sucrose tristearate); a preservative, e.g. phenoxyethanol and/or paraben and/or a paraben blend; and water; and optionally further comprising one or more of a humectant, e.g. glycerin; a fragrance; a pigment; and a color.

4. Cosmetic composition according to claim 1, wherein the cosmetic base is a gel comprising a thickener, e.g. xanthan gum; a preservative, e.g. phenoxyethanol and/or paraben and/or a paraben blend; and water; and optionally further comprising one or more of a humectant, e.g. glycerin, a fragrance, a pigment; and a color.

5. Cosmetic composition according to claim 1, wherein the cosmetic base is a jellified oil, comprising an emollient, e.g. coco-glycerides and/or octyldodecanol and/or C12-15 alkyl benzoate; a thickener, e.g. silica and/or cellulose and/or a cellulose derivative, such as ethyl cellulose, dextrin palmitate, dextrin myristate, dextrin palmitate ethylhexanoate, bentonite; and optionally further comprising one or more of xanthan gum; acrylate copolymers (e.g. carbomers); a fragrance; a pigment; and a color.

6. Cosmetic composition according to claim 1, wherein the cosmetic base is a shampoo base comprising a surfactant, e.g. sodium laureth sulfate and/or cocamidopropyl betaine; a preservative, e.g. phenoxyethanol; and water; and optionally further comprising one or more of a conditioner, e.g. hydroxypropyl guar and/or hydroxypropyl guar hydroxypropyltrimonium chloride; a pH modulator, e.g. an acid, such as citric acid, or a base; a viscosity controller and/or bulking agent, e.g. sodium chloride or another salt; a fragrance; a pigment; and a color.

7. Cosmetic composition according to claim 1, wherein the cosmetic base is a shampoo base comprising a surfactant, e.g. coco-glucoside and/or decyl-glucoside and/or a quillaja saponaria wood extract; an emollient, e.g. glyceryl oleate; a preservative, e.g. sodium benzoate and/or potassium sorbate; and water; and optionally further comprising one or more of a conditioner, e.g. xanthan gum; a pH modulator, e.g. an acid, such as lactic acid, or a base; a fragrance; a pigment; and a color.

8. Cosmetic composition according to claim 1, wherein the cosmetic base is selected from the group consisting of:

| Base | Composition |
|---|---|
| Cream | 5% of a mixture of Ceteth-20, Cetyl Alcohol, Glyceryl |
| | Stearate, PEG-75 Stearate and Steareth-20 (e.g. Emulium |
| | Delta); 12% medium-chain triglycerides (e.g. Mygliol 812 |
| | N); 0.4% phenoxyethanol and/or paraben and/or a |
| | paraben blend (e.g. Phenonip XB); 1% glycerin; and |
| | 80.6% water |
| Gel | 1% xanthan gum; 3% glycerin; 0.5% phenoxyethanol (e.g. |
| | Phenoxetol); and 80.6% water |
| Jellified | 25.5% C12-15 alkyl benzoate (e.g. DUB B1215); 20% coco- |
| oil | glycerides (e.g. Myritol 331); 43.5% octyldodecanol (e.g. |
| | Eutanol G); 3% ethyl cellulose (e.g. Ethocel Std 100 |
| | Premium; and 7% silica (e.g. Aerosil 200) |
| Shampoo | 28.93% sodium laureth sulfate (e.g. SLES Texapon NSO 27% |
| | in water); 12% cocamidopropyl betaine (e.g. Dehyton |
| | Kcos 30% in water); 0.6% phenoxyethanol; 0.2% |
| | hydroxypropyl guar and/or hydroxypropyl guar |
| | hydroxypropyltrimonium chloride (e.g. Jaguar C162); |
| | 0.07% citric acid; 0.5% sodium chloride; 0.2% fragrance; |
| | and 56.5% water |
| Sulfate- | 1% xanthan gum (e.g. XGF FEDCS-PC); 0.2% coco- |
| free | glucoside (e.g. Plantacare 818 UP); 20% decyl-glucoside |
| shampoo | (e.g. Oramix NS 10); 7% coco-glucoside/glyceryl oleate |
| | (e.g. Lamesoft PO 65); 0.2% quillaja saponaria wood |
| | extract (e.g. Sapnov vegan); 0.3% sodium benzoate; 0.2% |
| | potassium sorbate; 2% lactic acid (e.g. Purac HS 90); and |
| | 62.1% water |

9. Cosmetic composition according to claim 1, wherein the shell layer comprises calcium alginate.

10. Cosmetic composition according to claim 1, comprising about 0.1 to about 10% of capsules, more preferably about 0.5 to about 5% of capsules, e.g. about 1% of capsules.

11. Cosmetic composition according to claim 1, wherein the capsules have an average diameter of about 70 to about 5000 μm, more preferably of about 1000 to about 3000 μm, most preferably about 1500 to about 2500 μm.

12. Cosmetic composition according to claim 1, wherein the cosmetic active compound constitutes about 20-50 wt % of the capsules.

13. Cosmetic composition according to claim 1, wherein the capsules are visible to the eye.

14. Cosmetic composition according to claim 1, wherein the shell layer is configured to break or rupture under a mechanical load.

15. Cosmetic composition according to claim 1, wherein the shell layer comprises a colorant and/or pigment selected from the group consisting of an anthocyanin (e.g. cyanidin, peonidin, malvidin, delphinidin, petunidin, pelargonidin), curcumin, chlorophyll, lycopene, beta-carotene, capsanthin, capsorubin, spirulina, zinc oxide, titanium dioxide, iron oxide, charcoal, and mixtures thereof.

16. Cosmetic composition according to claim 1, additionally comprising one or more further cosmetic actives.
